(19) United States
(12) Patent Application Publication
(10) Pub. No.: US 2003/0175444 A1
(43) Pub. Date: Sep. 18, 2003

(54) Method for forming a tioss(2-x) film on a material surface by using plasma immersion ion implantation and the use thereof

(76) Inventors: Nan Huang, Sichuan Province (CN); Ping Yang, Sichuan Province (CN); Yongxiang Leng, Sichuan Province (CN); Junying Chen, Sichuan Province (CN); Hong Sun, Sichuan Province (CN); Jin Wang, Sichuan Province (CN); Guojiang Wan, Sichuan Province (CN)

Correspondence Address: FINNEGAN, HENDERSON, FARABOW, GARRETT & DUNNER, LLP, 1300 I STREET, NW, WASHINGTON, DC, 20005, US

(21) Appl. No.: 10/168,500

(22) PCT Filed: Dec. 25, 2000
(86) PCT No.: PCT/CN00/00728

(30) Foreign Application Priority Data
Dec. 23, 1999 (CN) 99117468.2

(51) Int. Cl. C23C 16/00; H05H 1/24; B05D 3/02; A61L 2/00

(52) U.S. Cl. 427/523; 427/569; 427/376.2; 427/255.28; 427/2.24

(57) ABSTRACT

The present invention discloses at least one to omnidirectionally modify surfaces of organic or inorganic materials by means of plasma immersion ion implantation process to produce TiO₂₋ₓ films of the materials surfaces (x is about 0˜0.35). The method includes using oxygen which exists as plasma in the PIII vacuum chamber as the environment, creating and introducing titanium and other metallic plasmas which deposit on the materials surfaces, into the vacuum chamber by means of metal arc source, and apply a negative pulse potential of 500˜50,00 Hz and 0.1˜10 kV amplitude on the workpiece. And also the method to by means of implanting H, Ta or Nb into the TiO₂₋ₓ films to produce TiO₂₋ₓ films containing H, Ta or Nb. The materials with surface films fabricated by the present invention, when implanted into human body and contacting blood, have obvious improved blood compatibilities.

The present invention relates to techniques for material surface modification, more precisely, to the methods of surface modification of inorganic and organic materials The present invention is also related to a surface modification method for materials of artificial organs, especially for the artificial organs and the implants which contact with blood.

Biocompatibility and durability are the basic requirements for applications of artificial organs. For artificial cardiovascular devices, such as artificial heart, artificial heart valve and left ventricular pump, even higher biocompatibility and durability are required. De Yong Hao Yi et al. described the state of application of artificial organs: The artificial heart and artificial heart valves made up of nature materials such as porcine pericardium and bovine pericardium and polymer materials cannot meet the durability requirement. While for the artificial heart valves made up of inorganic materials such as low temperature isotropic pryolytic carbon (LTIC), titanium alloy, cobalt alloy and stainless steels, the two problems existed: the first is poor blood compatibility, and the second is failure due to fatigue, corrosion, wear off and crack after the artificial heat valves are implanted into a human body (The State of Frequently Applied Artificial Organs and the Future—Artificial Heart Valves, The Journal Artificial Oranges, 1990, 19(3):pp100-102). LTIC is the best material in blood compatibility and represents the highest level of artificial heart valves used in clinic. However, for the requirement for clinic applications, its blood compatibility is still not good enough, while its toughness is only one percent of that of metals. Paul Didisheim mentioned that the incidence of thromboembolic complications and bleeding is 1.5-3.0 present/year for each complications in the United States ( Substitute Heart Valves—Do We Need Better Ones, Government News, Biomaterials Forum, 1996,18(5), pp15-16). It is necessary to develop new artificial heart valves which are superior in anticoagulation properties to the valves used now. New materials, surface modifications and new valve designs are approaches to develop new heart valves. Mitamura Y. et al described a method to coat a titanium nitride film on titanium artificial heart valve by means of physical vapor deposition, and mentioned that the blood compatibility of the titanium nitride film is similar to LTIC (Journal of Biomaterials Applications, 1989,4(11), pp33-55). At present, two problems exist with coatings of titanium nitride film, diamond like carbon film, and LTIC film on materials for artificial cardiovascular system: 1)The coating does not improve blood compatibility significantly. The blood compatibility of coated materials is not obviously superior to the presently used LTIC; 2)The adhesion strength between the coated films and substrate material is low due to the limitation of the technique. Chinese patent ZL 95111386.0 disclosed a method using ion beam enhanced deposition to synthesize titanium oxide/titanium nitride multi-layer films on cardiovascular artificial organs. The method can only be used to modify components with a plane or simple shape, such as leaflets of artificial heart valves, but cannot be used with artificial organs of complicated shapes (such as the cage of artificial heart valves). However, to obtain the required properties and safety of an artificial organ, all surfaces of the artificial organs contacting blood need to be thoroughly and homogeneously modified,

As said above, a surface modification method is needed to improve the blood compatibility of inorganic and organic materials, so when the material is used with artificial organs and devices implanted into human body, the material will have good blood compatibility.

A purpose of the present invention is to develop surface modification materials and the method.

Another purpose of the present invention is to develop surface modification materials for artificial organs and the method.

The purpose of the present invention is also to develop surface modification materials for artificial organs and devices which are implanted into human bodies and contact blood, and the method. The present invented method can be used to significantly improve the blood compatibility of the surfaces for artificial heart, artificial heart valves, left ventricular pump, vascular stents and other cardiovascular devices which have complicated shapes.

The present invention uses a special technique to prepare a two-component titanium oxide film, and multi-component titanium oxide films containing hydrogen, tantalum or niobium, and first to obtain a titanium nitride film, and then on it prepare a layer with a gradient composition decreasing in nitrogen content and increasing in oxygen content, for the purpose to fabricate the material surface with excellent blood compatibility and good mechanical properties. The present invention can be realized by the following methods: (Hereinafter, the term “workpiece” includes the term “artificial organs” and the “devices” implanted into human body and contacting blood; also, the term “artificial organs” or “devices” can be understood to be any inorganic or organic materials used in any other field.)

1. Synthesis of TiO₂₋ₓ Film on Material Surface with Oxygen Vacancy and TiO₂₋ₓ/Ti—N—O/TiN Gradient Film

(1) TiO₂₋ₓ Film with Oxygen Vacancy

Place the workpieces (such as an artificial organs) on the work stage of the vacuum chamber of a plasma immersion ion implantation equipment (PIII). Back fill oxygen into the vacuum chamber to a certain pressure. Using radio frequency discharge (RF) or microwave (MW) discharge to generate oxygen plasma. In the mean time, use titanium as the cathode of the metal plasma source of PIII. Turn on the titanium plasma source and introduce the titanium plasma into the vacuum chamber. Under the pulse negative potential on the workpieces, the titanium and oxygen ions bombard on the workpieces (artificial organs) surface and form a TiO₂₋ₓ film. The factors controlling the film properties are: density of the titanium plasma, The deposition rate of titanium ions, the density of oxygen plasma, oxygen pressure, frequency of the pulse negative potential, pulse width and amplitude of the pulse negative potential.

(2) Synthesis of TiO₂₋ₓ/Ti—N—O/TiN Gradient Film

Place the workpieces (artificial organs) in the vacuum chamber of PIII. Back fill nitrogen into the chamber to a certain pressure. Use RF discharge or MW discharge to generate nitrogen plasma. In the mean time, use titanium as the cathode of the metal plasma source to produce titanium plasma. Turn on the metal plasma source and introduce titanium plasma into the vacuum chamber. Under the pulse negative potential on the workpieces, titanium and nitrogen ions bombard on the workpieces (artificial organs) surface and form a TiN film. Then, gradually decrease the nitrogen pressure and increase oxygen pressure in the vacuum chamber. A gradient Ti—N—O film with a decreasing nitrogen content and increasing oxygen content can be formed. After certain time, there is only oxygen in the vacuum chamber, except the titanium plasma. The oxygen and oxygen plasma, and titanium plasma will form a TiO₂₋ₓ film under the effect of the pulse negative potential. The factors controlling the properties of the film include: the density of the titanium plasma, the deposition rate of titanium ions, the density of nitrogen plasma, the density of oxygen plasma, nitrogen pressure, oxygen pressure, the repeated frequency of the negative pulse potential, pulse width and amplitude of the negative pulse potential.

Using the method described in above terms (1) and (2), a TiO2-x film with oxygen vacancy can be obtained on the workpieces surface. The workpiece can be annealed at certain temperature for certain time in vacuum. The factors controlling the properties of the film after annealing are: annealing temperature, annealing time and vacuum pressure.

2. Synthesis of Ti—O Film Containing Hydrogen on a Material Surface

A titanium oxide film containing hydrogen on a material surface can be synthesized by the following technique.

Place the workpieces (artificial organs) in the vacuum chamber of PIII. Back fill the chamber with oxygen to a certain pressure. Use RF or MW discharge to create oxygen plasma, in the mean time use titanium as the cathode of the metal plasma source to produce titanium plasma. Turn on the metal plasma source and introduce the titanium plasma into the vacuum chamber. Under the pulse negative potential on the workpieces, titanium and oxygen ions bombard on the workpieces (artificial organs) surface and form a TiO₂ film. The factors controlling the properties of the film are: the density of the titanium plasma, the deposition rate of the titanium ions, the density of the oxygen plasma, oxygen pressure, the repeat frequency of the pulse negative potential, pulse width and amplitude of the pulse negative potential.

(1) Plasma Hydrogenation

Place the workpieces (artificial organs) with a TiO₂ film in the vacuum chamber of PIII. Fill the chamber with hydrogen to a certain pressure. Use electric discharge to create hydrogen plasma and apply a pulse or direct negative potential on the workpieces. (the workpieces (artificial organs) can be heated at the same time).

A TiO₂ film containing hydrogen can be fabricated. The factors controlling the properties of the film are: the hydrogen pressure, the density of the hydrogen plasma, heating temperature, electric voltage and current for discharge, and the time of hydrogenation treatment.

(2) Hydrogenation by Single Ion Implantation

Place the workpieces (artificial organs) with a TiO₂ film in the vacuum chamber of PIII. Fill the chamber with hydrogen to a certain pressure. Use RF or MW discharge to create hydrogen plasma and apply a pulse negative potential on the workpieces (artificial organs). Hydrogen will be implanted into the TiO₂ film. A modification layer of Ti—O containing hydrogen can be fabricated. The factors controlling the properties of the film are the hydrogen pressure in the vacuum chamber, the density of the hydrogen plasma, the energy of hydrogen ions, the dosage of hydrogen ions, the repeat frequency of the pulse negative potential., pulse width and amplitude of the pulse negative potential.

(3) Hydrogenation by Multi-Ion Implantation

Place the workpieces (artificial organs) with a TiO₂ film surface in the vacuum chamber of PIII. Fill the chamber with hydrogen to a certain pressure and create hydrogen plasma. Implant hydrogen ions into the film at pulse negative high potential. After certain time, decrease the potential and implant hydrogen by the same technique. Then, after certain time decrease the potential again. Repeat this process to get a film with a homogeneous distribution profile of hydrogen. The factors controlling the properties of the film are: the hydrogen pressure in the vacuum chamber, the density of the hydrogen plasma, the energy of hydrogen ions, the dosage of hydrogen ions, the repeat frequency of the pulse negative potential, pulse width and amplitude of the pulse negative potential, the repeating times of ion implantation and the time for each implantation.

The workpieces (artificial organs) treated using the techniques described above can be annealed in vacuum to get a Ti—O film containing hydrogen and with excellent property. The factors controlling the film are annealing temperature, time and vacuum pressure.

It is also possible to fabricate a titanium nitride film first, then fabricate a gradient TiO₂/Ti—N—O/TiN film with decreasing nitrogen content and increasing oxygen content, and finally to fabricate the hydrogen doped Ti—O surface film.

3. Doping Niobium or Tantalum into TiO₂ Film

The following methods can be used to make Ti—O film containing niobium or tantalum.

1) Synthesize TiO₂ Film or TiO₂/Ti—N—O/TiN Gradient Film Containing Tantalium or Niobium Using PIII

(a) Ion Implatation

First prepare a TiO₂ film or TiO₂/Ti—N—O/TiN gradient film on the surface of workpieces using PIII. Put the workpieces with the prepared film on the work stage in the vacuum chamber of PIII and apply a pulse negative potential. Use tantalum or niobium as the cathode of the metal plasma source of PIII. Turn on the metal plasma source and introduce the tantalum or niobium plasma into the vacuum chamber. Under the attraction of the high negative pulse potential, the tantalum or niobium ions bombard and implant into the workpieces surface, and to form tantalum or niobium doped Ti—O film, and to form tantalum or niobium doped TiO₂ or TiO₂/Ti—N—O/TiN gradient film. A homogeneous profile of tantalum or niobium content can be achieved by changing the potential on the workpieces and treat the workpieces repeatedly. The factors controlling the properties of the film are: the density of tantalum or niobium plasma, the implantation dosage of tantalum or niobium, the repeat frequency of the pulse negative potential, pulse width and amplitude of the pulse negative potential, and the times of changing the potential amplitude.

(b) Film Deposition

Place the workpieces (artificial organs) on the work stage in the vacuum chamber of PIII. Fill the vacuum chamber with oxygen to a certain pressure. The oxygen in the chamber can be either neutral gas, or can be transformed to plasma by RF or MW discharge. Apply a negative potential on the workpieces, turn on the metal plasma source of PIII. The cathode of the metal plasma is a Ti—Ta or Ti—Ni alloy. Introduce the metal plasma into the vacuum chamber. Under the attraction of the pulse negative potential, Ti, Ta or (Nb) and oxygen ions will bombard on the workpieces (artificial organs) surface and form a Ti—O film containing tantalum (or niobium). The factors controlling the properties of the film are: The ratio of Ta/Ti or (Nb/Ti) ion in the Ti—Ta (Ti—Nb) plasma, the density of Ti and Ta (or Ti and Nb) plasma, the density of oxygen plasma, the oxygen pressure, the repeat frequency of the pulse negative potential, pulse width and amplitude of the pulse negative potential.

It is also possible to introduce tantalum (or niobium) plasma, titanium plasma and nitrogen plasma (or nitrogen atmosphere) into the vacuum chamber and fabricate a titanium nitride film containing tantalum (or niobium first, then decrease the nitrogen content but increase oxygen content, to fabricate a gradient TiO₂/Ti—N—O/TiN film with decreasing nitrogen content and increasing oxygen content.

Using the methods described in (a), (b) above, a Ti—O or TiO₂/Ti—N—O/TiN gradient film doped with tantalum (or niobium) can be produced on the artificial organs surface. The artificial organs can be also annealed in vacuum for some time at certain temperature. The factors controlling the properties of the film will be annealing temperature, annealing time and vacuum pressure.

2) Synthesize TiO₂ Film Containing Tantalum or Niobium Using Magnetron Sputter-Ion Coating

(1) First, use a Ti—Ta (or Ti—Nb) alloy or Ti metal embedded with tantalum (or niobium) as the target material, utilize magnetron sputter of high speed low temperature deposition method to prepare. Ti—Ta (or Ti—Nb) alloy film. Apply a direct or pulse negative potential on the target. Introduce argon into the vacuum chamber of the magnetron sputter device and create argon plasma. The argon plasma will bombard on the target. The atoms sputtered out from the target will deposit on the workpieces (artificial organs) which is in rotation movement in the vacuum chamber. The factors controlling the properties of the film are the ratio of Ta/Ti (or Nb/Ti) in the alloyed target material or embedded target material, the sputter potential (direct or pulse), the sputter powder density, heating temperature, time for sputter treatment, potential on the workpiece (direct or pulse), the argon pressure in the vacuum chamber, and the rotation speed of the workpieces.

(2) Introduce argon and nitrogen together into the vacuum chamber of the magnetron sputter device. The sputtered atoms from the target will react with nitrogen and form a titanium nitride film containing tantalum (or niobium). The factors controlling the properties of the film are: The ratio of Ta/Ti (or Nb/Ti) in the alloyed target material or embedded target material, the sputter potential (direct or pulse), the sputter power density, the heating temperature, the sputter time, the sputter pressure, the potential on the workpieces (direct or pulse), and the pressure of argon and nitrogen. Titanium nitride film contain Ta or Nb can be obtained.

A titanium oxide or titanium oxide/nitride film containing tantalum (or niobium can be obtained by oxidizing the films prepared using above mentioned methods (1) and (2). The oxidization treatment can be done by two methods, as below:

A. Thermal Oxidation

Put the workpieces (artificial organs) with Ti—Ta or Ti—Nb coated surface in a quartz tube, Heat the quartz tube to certain temperature and back fill oxygen to a certain pressure. The film will be oxidized and transform to a titanium oxide film, containing tantalum (niobium). The factors controlling the properties of the film are: oxygen pressure, heating temperature, time for oxidization treatment, the content of tantalum (or niobium) in the Ti—Ta (or Ti—Nb) film.

B. Plasma Oxidation

Put the workpieces (artificial organs) with Ti—Ta or Ti—Nb coated surface in the vacuum chamber of PIII and fill the vacuum chamber with oxygen to certain pressure. Create oxygen plasma using RF and MW discharge. Now the workpieces are immersed in an oxygen plasma environment. Heat the workpieces and apply a direct of pulse negative potential. The oxygen atoms will bombard on the workpieces surface and form a titanium oxide film, containing tantalum (or niobium). The factors controlling the properties of the film are: The oxygen pressure, the density of oxygen plasma, the heating temperature, the amplitude of the negative potential applied on the artificial organs, the treatment time of plasma oxidation, the repeat frequency of the negative potential, pulse width of the negative potential, and the composition of the Ti—Ta (or Ti—Nb) film.

(3) Apply a negative pulse potential on the sputter target and fill the vacuum chamber with argon and oxygen together. Atoms of the target will be sputtered out and will react with oxygen and form a titanium oxide film, containing tantalum (or niobium). The factors controlling the properties of the film are: the atomic ratio of tantalum (or niobium) to titanium in the alloyed target material or embedded target material, the pulse sputter potential, the sputter powder density, frequency and width of the pulse sputter potential, the heating temperature on the workpieces, the sputter time, the pulse potential applied on the sample stage, the frequency of and the width of the potential on sample stage, the argon and oxygen pressure, and the rotation speed of the workpieces.

It is also possible to fill the vacuum chamber with argon and nitrogen first, and then decrease nitrogen pressure, but fill with oxygen with increasing pressure, thus to obtain a film of TiO₂/Ti—N—O/TiN containing Ta or Nb and with increasing oxygen content and decreasing nitrogen content.

(4) Use Ta₂O₅ (or Nb₂O₅) ceramic material as the target material. Fill the vacuum chamber with argon or xenon to a certain pressure, use RF discharge to create plasma. The plasma bombards the target. Atoms of the target material will be sputtered out and form Ti—O film, containing tantalum (or niobium) on the artificial organs surface. The factors controlling the properties of the film are: power and potential of the RF discharge, pressure of argon or xenon, potential of RF, temperature of the workpieces, the sputter time, composition of the Ta₂O₅ (or Nb₂O₅) ceramic target material, potential on the workpieces (pulse or direct), the rotation speed of the workpiece.

Comparing with other existing techniques, the present invention has advantages in: the TiO₂₋ₓ film and TiO₂/Ti—N—O/TiN gradient film containing hydrogen, tantalum (or niobium) obtained by the present invention has a very good blood compatibility. The blood compatibility of the film is significantly superior to low temperature isotropic pryolitic carbon (LTIC) (which is recognized ad the best material for artificial heart valves so far). Homogeneous coating film on artificial organs with complicated shapes can be realized. The film can be produced in industrial scale. The composition of the film can be easily controlled and has a high repeatability. The binding strength of the film to the substrate is strong. By using the prevent invention, the blood compatibility , corrosion resistance and wear resistance of the workpieces can all be improved.

### BRIEF DESCRIPTION OF THE DRAWINGS

FIG. 1 schematiclly illustrates a PIII system used in the present invention.

FIG. 2 schematiclly illustrates a RF magnetron sputter device used in the present invention.

FIG. 3. The vacuum quartz tube furnace used in the present invention.

FIG. 4 shows the in vitro test results of platelet adhesions on films synthesized by the present invention (a) and on TLIC (b).

FIG. 5 shows the results (a) Blood cell adhesion on a film synthesized by the present invention after in vivo test, (b) and (c) Blood cell adhesion on the surface of LTIC after in vivo test.

FIG. 6 Shows the results (a) Thrombus on the surface of an commercial artificial heart valve cage modified using the present invention after in vivo test; (b) Thrombus on the surface of an artificial heart valve cage without surface modification after in vivo test.

FIG. 7 shows the results of comparison in wear resistances of titanium materials for artificial heat valves with and without surface modification using the present invention.

### FURTHER DESCRIPTION OF THE INVENTION

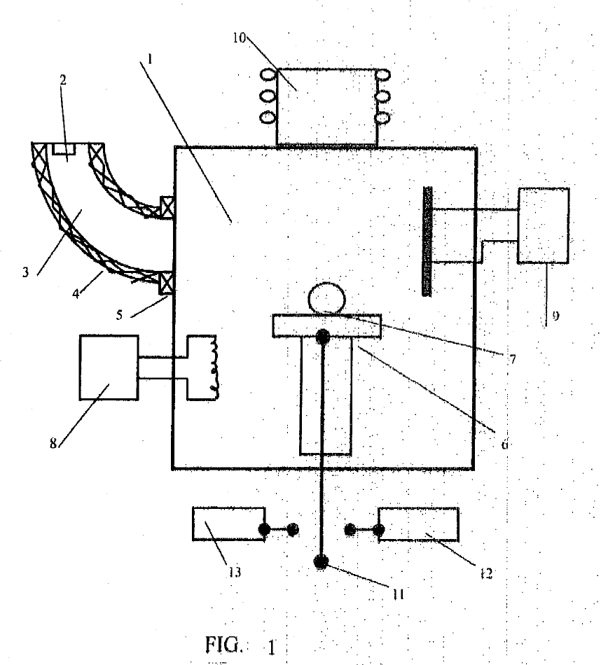

Referring to FIG. 1, a PIII equipment includes: vacuum chamber 1, cathode 2, negative arc plasma source 3, bias winding 4, scanning coil 5, work stage 6, workpiece (artificial organ or other organic or inorganic material) 7, power source for the filament 8, power for RF discharge 9, power for MW discharge 10, switch 11, direct potential power 12 and pulse high voltage power 13. Using this equipment, TiO₂₋ₓ films, TiO₂₋ₓ/Ti—N—O/TiN gradient films, Ti—O films containing hydrogen, tantalum or niobium, can be made.

1. Synthesis of TiO₂₋ₓ or TiO₂₋ₓ/Ti—N—O/TiN Gradient Films

### EXAMPLE 1

Referring FIG. 1, install workpieces (artificial organs) 7 on the work stage 6 in vacuum chamber 1 of the PIII equipment. Install a titanium cathode 2 on the metal negative arc plasma source 3. Pump out air in the vacuum chamber 1. When the pressure in the vacuum chamber reached 1×10⁻⁴ Pa, fill the vacuum chamber 1 with oxygen. Turn on the power of RF discharge 10 (or power of MW discharge) to create oxygen plasma. Turn on switch 11 to pulse potential 13 and apply a pulse negative potential on the work stage. Open the cathode arc plasma source 3. Turn on the bias magnetic winding 4 and scanning coil 5 outside the metal cathode arc plasma source and introduce the titanium plasma into the vacuum chamber. Under the attraction of the negative pulse potential, titanium and oxygen ions will bombard the workpiece (ar4tifical organ) 7 at the same time and form a titanium oxide film of the workpiece surface. The film can be formed using parameters given in Table 1. The factors controlling the properties of the film are: the density of titanium plasma 10⁸˜10¹² cm⁻³, the density of oxygen plasma 10⁸˜10¹² cm⁻³, the titanium deposition rate on the workpiece surface 0.1˜1 nm/s, oxygen pressure 10⁻³˜1 Pa, the repeat frequency of the negative potential 500˜5000 Hz, pulse width 1˜200 μs, the amplitude of the pulse negative potential 0.1˜10 kV.

TABLE 1

| | Density of | Rate of Ti | Density of | Oxygen | Pulse | Pulse | Pulse |
|---|---|---|---|---|---|---|---|
| | Ti piasma | deposition | oxygen plasma | pressure | frequency | width | potential |
| Condition | (cm⁻³) | (nm/s) | (cm⁻³) | (Pa) | (Hz) | (μs) | (kV) |
| 1 | 3 × 10⁸ | 0.08 | 5 × 10⁸ | 5 × 10⁻³ | 500 | 20 | −0.1 |
| 2 | 3 × 10⁹ | 0.2 | 5 × 10⁹ | 1.6 × 10⁻² | 25000 | 5 | −2.5 |
| 3 | 3 × 10¹⁰ | 1 | 5 × 10¹⁰ | 6 × 10⁻² | 2000 | 100 | −10 |

### EXAMPLE 2

Referring FIG. 1, install workpiece 7 on the work stage 6 in vacuum chamber 1 of the PIII equipment. Install a titanium cathode 2 on the metal cathode arc plasma source 3. Pump out air in the vacuum chamber 1. To produce a nitride film first, when the pressure in the vacuum chamber reached 1×10⁻⁴ Pa, back fill the vacuum chamber 1 with nitrogen. Turn on the RF discharge power 10 (or MW discharge power) to create nitrogen plasma. Turn on switch 11 to the position of pulse high potential 13 and apply a pulse negative potential on the work stage. Open the cathode arc plasma source 3. Turn on the bias winding 4 and scanning coil 5 outside the metal cathode arc plasma source and introduce the titanium plasma into the vacuum chamber. Under the attraction of the negative potential, titanium and nitrogen ions will bombard the artificial organ 7 at the same time and form a titanium nitride film on the artificial organ surface. Then, introduce oxygen into the vacuum chamber. Decrease the nitrogen pressure and increase the oxygen pressure gradually to form Ti—N—O middle layer with nitrogen atom decrease and oxygen atom increase. At the final stage, there will be only oxygen and titanium plasmas in the vacuum chamber, and TiO₂₋ₓ film is formed on the top layer. The gradient film TiO2-x/Ti—N—O/TiN can be formed using parameters given in Table 2. The factors controlling the properties of the film are the density of titanium plasma 10⁸˜10¹² cm⁻³, the density of nitrogen plasma 10⁸˜10¹² cm⁻³, the density of oxygen 10⁸˜10¹² cm⁻³, the titanium deposition rate on the artificial organs surface 0.1˜1 nm/s, nitrogen pressure 10⁻³˜1 Pa, oxygen pressure 10⁻³˜1 Pa, the repeat frequency of the negative pulse potential 500˜50,000 Hz, pulse width 1˜200 μs, the amplitude of the pulse negative potential 0.1˜10 kV, the changing rate of nitrogen and oxygen pressure in the vacuum chamber is 10⁻³˜10⁻² Pa/min.

The workpiece treated by example 1 or 2 stated process can be annealed in vacuum chamber of PIII equipment by heating the workpieces on the work stage of 100˜800° C. for 0.1˜2 hour at 10^{-4˜10}⁻¹ Pa. After annealing, the value of x for TiO₂₋ₓ film is 0.05˜0.35, the typical structure of the film is rutile, the thickness of the film is 0.05˜5 μm, the thickness of gradient layer is 10˜100 nm.

TABLE 2

| | | |
|---|---|---|
| 1^{st} stage | 2^{nd} stage | 3^{rd} stage |

| | | | Density of | | Nitrogen | Oxygen | Density of | | | | |
|---|---|---|---|---|---|---|---|---|---|---|---|
| | Density of | Rate of Ti | nitrogen | Nitrogen | pressure | pressure | oxygen | Oxygen | Pulse | Pulse | Pulse |
| | Ti plasma | deposition | plasma | pressure | decrease rate | increase rate | plasma | pressure | frequency | width | potential |
| Condition | (cm⁻³) | (nm/s) | (cm⁻³) | (Pa) | (Pa/s) | (Pa/s) | (cm⁻³ | (Pa) | (Hz) | (μs) | (kV) |
| 1 | 3 × 10⁸ | 0.1 | 3 × 10⁸ | 5 × 10⁻³ | 1 × 10⁻³ | 1 × 10⁻³ | 5 × 10⁸ | 8 × 10⁻³ | 500 | 20 | −0.1 |
| 2 | 3 × 10⁹ | 0.2 | 5 × 10⁹ | 2 × 10⁻² | 2 × 10⁻³ | 2 × 10⁻³ | 4 × 10⁸ | 1 × 10⁻³ | 25,000 | 5 | −2.5 |
| 3 | 3 × 10¹⁰ | 1 | 5 × 10¹⁰ | 5 × 10⁻² | 3 × 10⁻³ | 3 × 10⁻³ | 2 × 10¹⁰ | 4 × 10⁻² | 2,000 | 100 | −10 |

2. Preparation of Titanium Oxide Film Containing Hydrogen

### EXAMPLE 3

Referring FIG. 1, install workpieces (artificial organs) 7 on the work stage 6 in vacuum chamber 1, of the PIII equipment. Prepare a TiO₂ film, or TiO₂/Ti—N—O/TiN gradient film on the artificial organs using the method described in Example 1 or Example 2. The processing parameters are given in Table 3.

TABLE 3

| | Density of | Rate of Ti | Density of | Oxygen | Pulse | Pulse | Pulse |
|---|---|---|---|---|---|---|---|
| | Ti plasma | deposition | oxygen plasma | pressure | frequency | width | potential |
| Condition | (cm⁻³) | (nm/s) | (cm⁻³⁾ | (Pa) | (Hz) | (μs) | (kV) |
| 1 | 3 × 10⁹ | 0.2 | 7 × 10⁹ | 2 × 10⁻² | 25,000 | 5 | −3.5 |

When the surface film is made, pump out the gas from the vacuum chamber of PIII. When the pressure in the vacuum chamber is below 10⁻³ Pa, back fill the vacuum chamber with hydrogen. Heat the artificial organs Turn on switch 11 to the position of low pulse potential 12 and apply −0.05˜−5 kV pulse potential. Turn on the powder of RF discharge 9 or MW discharge 10 to create hydrogen plasma. A Ti—O film, or TiO₂/Ti—N—O/TiN gradient film containing hydrogen can be made in 0.1˜2 hours. The hydrogenation can be performed using parameters given in Table 4. The factors controlling the properties of the film are: hydrogen pressure (10⁻³˜10 Pa), the density of hydrogen plasma (10^{8˜10}¹² cm⁻³), heating temperature 100˜600° C., discharge potential −0.2˜−5 kV, current 0.1˜5 A and processing time 0.1˜2 hour.

After hydrogenation, the workpiece can be annealed in vacuum for 0.1˜1 hour at 200˜600° C., Vacuum pressure 10⁻⁴˜10⁻¹ Pa. After annealing, the Ti—O film or TiO₂/Ti—N—O/TiN gradient film containing hydrogen is formed. The hydrogen content in the film is 10 at. %˜35 at. %.

TABLE 4

| | Hydrogen | Heating | | Density of | | |
|---|---|---|---|---|---|---|
| | pressure | temperature | Potential | hydrogen plasma | Current | Processing time |
| Condition | (Pa) | (° C.) | (kV) | (cm⁻³) | (A) | (hour) |
| 1 | 0.01 | 200 | −0.2. | 10⁸ | 0.1 | 2 |
| 2 | 0.1 | 300 | −0.8 | 5 × 10⁹ | 1 | 1 |
| 3 | 0.8 | 400 | −2 | 5 × 10¹⁰ | 3 | 0.5 |
| 4 | 10 | 500 | −3 | 10¹¹ | 5 | 0.1 |

### EXAMPLE 4

Referring FIG. 1, install a workpieces (artificial organs) 7 with TiO₂ film or TiO₂/Ti—N—O/TiN gradient film on the surface on the workstage 6 in the vacuum chamber of PIII shown in FIG. 1. Pump out air from the vacuum chamber. When the pressure in the vacuum chamber is below 10⁻⁴ Pa, back fill hydrogen into the chamber. Turn on switch 11 to the position of high voltage pulse potential 13 and apply a pulse negative potential on the artificial organs 7. Turn on the power of RF discharge 9 or MW discharge 10 to create hydrogen plasma and implant hydrogen ions into the artificial organs by PIII to form a hydrogen containing surface film. This process can be performed using parameters given in Table 5. The factors controlling the properties of the film are: hydrogen pressure 10⁻³˜10 Pa, the density of hydrogen plasma 10⁸˜10¹² cm⁻³, the hydrogen ion implantation dosage 10¹⁵˜1.2×10¹⁸ atom/cm², repeat frequency of the pulse negative potential 50˜20,000 Hz, pulse width 1˜200 μs and the amplitude of the pulse negative potential 1˜100 kV. After the treating by above process, the artificial organs 7 can be further annealed in vacuum chamber of FIG. 1 shown device for 0.1˜2 hour at 100˜400° C., Vacuum pressure 10⁻⁴˜10⁻¹ Pa. After annealing, the Ti—O film or TiO₂/Ti—N—O/TiN gradient film containing hydrogen is formed. The hydrogen content in the film is 10 at. %˜35 at. %.

TABLE 5

| | Hydrogen | Pulse | Pulse | Pulse | Density of | Hydrogen ions |
|---|---|---|---|---|---|---|
| | pressure | Potential | width | frequency | hydrogen | implantation |
| Condition | (Pa) | (kV) | (μs) | (Hz) | plasma (cm⁻³) | dosage (atom/cm⁻³) |
| 1 | 0.001 | −2 | 2 | 20,000 | 10⁸ | 2 × 10¹⁶ |
| 2 | 0.017 | −20 | 3 | 200 | 2 × 10⁹ | 1.5 × 10¹³ |
| 3 | 0.3 | −50 | 40 | 1,000 | 6 × 10¹⁰ | 5 × 10¹⁷ |
| 4 | 1 | −100 | 200 | 200 | 10¹² | 9 × 20¹² |

After hydrogenation treatment as described above, the artificial organs can be annealed in vacuum, in the condition given in Table 6. The annealing can be carried out in the vacuum chamber of PIII equipment by heating the artificial organs on the work stage to 100˜400° C. for 0.1˜2 hour at 10⁻⁴˜10⁻¹ Pa. The annealing parameters are given in Table 6. After annealing, the TiO₂₋ₓ film or TiO₂/Ti—N—O/TiN gradient film will contain 10˜35 at. % hydrogen.

TABLE 6

| Condition | Tempergtnre ° C. | Processing time (min.) | Pressure (P₄) |
|---|---|---|---|
| 1 | 230 | 55 | 2 × 10⁻³ |
| 2 | 280 | 65 | 8 × 10⁻⁴ |

### EXAMPLE 5

Referring FIG. 1, install the workpieces (artificial organs) 7 with TiO₂₋ₓ film or TiO₂/Ti—N—O/TiN gradient film on workstage 6 in the vacuum chamber of PIII equipment as shown in FIG. 1. Pump out air from the vacuum chamber. When the pressure in the vacuum chamber is below 10⁻⁴ Pa, back fill hydrogen into the chamber. Turn on switch 11 to the position of high voltage pulse potential 13 and apply a 70˜100 kV pulse negative potential on the workpiece 7. Turn on the power of RF discharge 9 or MW discharge 10 to create hydrogen plasma, and implant hydrogen into the workpieces by PIII technique. After 0.1˜2 hour, decrease the pulse potential to apply 30˜60 kV potential on the workpieces and implant hydrogen into the workpiece for certain time. Then decrease the potential again. Repeat this process to implant hydrogen into the workpieces, to get a Ti—O film or TiO₂/Ti—N—O/TiN gradient film with homogeneously distributed profile of hydrogen. This process can be performed using parameters given in Table 7. The factors controlling the properties of the film are: hydrogen pressure 10⁻²˜1 Pa, the density of hydrogen plasma 10⁸˜10¹² cm⁻³, the hydrogen ion implantation dosage 10¹⁶˜10¹⁸ atom/cm², repeat frequency of the pulse potential 50˜20,000 Hz, pulse width 2˜200 μs, the amplitude of the negative pulse potential 1˜100 kV, repeat treatment 2˜10 times and the processing time for each treatment 0.1˜2 hour.

TABLE 7

| | | Amplitude | | Amplitude | | Amplitude | | Amplitude | | | | Density of | Hydrogen |
|---|---|---|---|---|---|---|---|---|---|---|---|---|---|
| | Hydrogen | of Potential | | of Potential | | of Potential | | of Potential | | Pulse | Pulse | hydrogen | implantation |
| | pressure | and working | | and working | | and working | | and working | | width | frequency | plasma | dosage |
| Condition | (Pa) | time | | time | | time | | time | | (μs) | (Hz) | (cm⁻³) | (atom/cm⁻³) |
| 1 | 2 × 10⁻² | 7 | kV and | 18 | kV and | | | | | 30 | 500 | 2 × 10⁸ | 2.1 × 10¹³ |
| | | 1.5 | hour | 2 | hour | | | | | | | | |
| 2 | 1.7 × 10⁻² | 7 | kV and | 15 | kV and | 35 | kV and | 55 | kV and | 5 | 250 | 6 × 10⁸ | 5.6 × 10² |
| | | 0.25 | hour | 0.7 | hour | 1 | hour | 1 | hour | | | | |
| 3 | 4 × 10⁻² | 10 | kV and | 30 | kV and | 60 | kV and | 95 | kV and | 100 | 50 | 2 × 10¹⁰ | 1 × 10¹⁰ |
| | | 0.05 | hour | 0.15 | hour | 0.2 | hour | 0.3 | hour | | | | |

After hydrogenation treatment as described above, the workpieces can be annealed in vacuum. The annealing can be carried out in the vacuum chamber of PIII equipment by heating the workpieces to 100˜400° C. for 0.1˜2 hour at 10⁻⁴˜10⁻¹ Pa. After annealing, the TiO₂₋ₓ film or TiO₂/Ti—N—O/TiN gradient film will contain 10˜35 at. % hydrogen.

3. Synthesis of Titanium Oxide Film Containing Niobium or Tantalum

1) TiO₂ Film Containing Nb or Ta can be Prepared Using PIII by the Following Approaches

### EXAMPLE 6

Referring FIG. 1, install workpieces (artificial organs) 7 with TiO₂ film on workstage 6 in the vacuum chamber of PIII equipment, and use a piece of tantalum (or niobium) as the cathode 2 mounted in metal cathode arc source 3. Pump out air from the vacuum chamber. When the pressure in the vacuum chamber is below 10⁻⁴ Pa, turn on switch 11 to the position of pulse high potential 13 and apply a pulse negative potential on the work stage. Turn on the cathode metal arc plasma source. Turn on the bias winding 4 and scanning coil 5 outside the metal cathode arc plasma source and introduce tantalum (or niobium) plasma into the vacuum chamber. Under the attraction of the pulse negative potential, tantalum (or niobium) ions will bombard and implant into the workpieces 7. This process can be performed using parameters given in Table 8. The factors controlling the properties of the film are: the density of tantalum (or niobium) plasma 10⁸˜10¹² cm⁻³, the ion implantation dosage of tantalum (or niobium) 10¹⁵˜5×10¹⁷ atom/cm², repeat frequency of the pulse negative potential 100˜20,000 Hz, pulse width 1˜200 μs, the amplitude of the negative pulse potential 1˜100 kV. After the film is produced, the workpieces can be annealed in the vacuum chamber of the PIII equipment by heating the workpieces to 100˜800° C. for 0.1˜2 hours, at pressure 10⁻⁴˜10⁻¹ Pa.

TABLE 8

| | Density of Ta | Implantation dosage | Pulse | | |
|---|---|---|---|---|---|
| | (or Nb) plasma | of Ta (or Nb) | frequency | Pulse width | Pulse potential |
| Condition | (cm⁻³) | (cm⁻²) | (Hz) | (μs) | (kV) |
| 1 | 1 × 10⁸ | 3.5 × 10¹⁵ | 20 | 5 | +20 |
| 2 | 5 × 10⁹ | 8 × 10¹⁵ | 500 | 100 | −50 |
| 3 | 1 × 10¹¹ | 1.5 × 10¹⁶ | 5,000 | 50 | −100 |
| 4 | 2 × 10¹⁰ | 1.2 × 10¹⁵ | 20,000 | 2 | 5 |

### EXAMPLE 7

Referring FIG. 1, install workpieces (artificial organs) 7 on workstage 6 in the vacuum chamber of PIII equipment. Use a piece of Ti—Ta (or Ti—Nb) alloy as the cathode material 2 on the cathode arc source 3. Pump out air from the vacuum chamber. When the pressure in the vacuum chamber is 1×10⁻⁴ Pa, back fill the vacuum chamber with oxygen to a certain pressure. Turn on switch 11 to the position of high pulse potential 13 and apply a pulse negative potential on the work stage. Turn on the cathode metal arc plasma source 3. Turn on the bias winding 4 and scanning coil 5 outside the metal cathode arc plasma source and introduce Ti—Ta (or Ti—Nb) plasma into the vacuum chamber. Under the attraction of the negative potential, Ti, Ta and oxygen (or Ti, Nb and oxygen) ions will bombard on the artificial organs surface and form a titanium oxide film containing tantalum (or niobium). This process can be performed using parameters given in Table 9. The factors controlling the properties of the film are: the atomic ratio of Ta/Ti (or Nb/Ti) is 0.5/100˜10/100, the density of Ti and Ta (or Ti and Nb) plasma 10⁸˜10¹² cm⁻³, the density of oxygen plasma 10⁸˜10¹² cm⁻³, oxygen pressure 10⁻³˜10 Pa, repeat frequency of the pulse potential 100˜20,000 Hz, pulse width 1˜200 μs, the amplitude of the pulse negative potential 0.10˜20 kV. After the film is produced, the workpieces can be annealed in the vacuum chamber of the PIII equipment by heating the workpieces to 100˜800° C. for 0.1˜2 hours, at pressure 10⁻⁴˜10⁻¹ Pa.

TABLE 9

| | Ta (Nb) | Deposition | Density of | | | | | |
|---|---|---|---|---|---|---|---|---|
| | content in | rate of metal | metal | Density of | Oxygen | Pulse | Pulse | Pulse |
| | the cathode | atoms | plasma | oxygen | pressure | frequency | width | potetial |
| Condition | (at. %) | (nm/s) | (cm⁻³) | plasma (cm⁻³) | (Pa) | (Hz) | (μs) | (kV) |
| 1 | 0.8 | 0.08 | 1 × 10⁹ | 2 × 10⁹ | 1.1 × 10⁻² | 10,000 | 10 | −0.5 |
| 2 | 3 | 0.2 | 6 × 10⁹ | 1 × 10¹⁰ | 2 × 10⁻² | 20,000 | 2 | −3 |
| 3 | 8 | 1 | 2 × 10¹⁰ | 3.5 × 10¹⁰ | 7 × 10⁻² | 50 | 200 | −10 |

2. Synthesize TiO₂ Film Containing Tantalum or Niobium Using Magnetron Sputter-Ion Coating

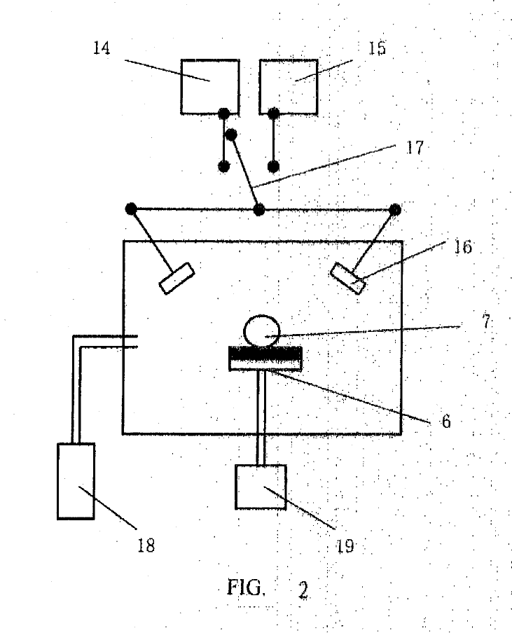

TiO₂ film containing tantalum or niobium can be prepared using magnetron sputter-ion coating technique. FIG. 2 is the schematic drawing of the magnetron sputter-ion coating equipment used in the present invention. The magnetron sputter-ion coating equipment includes: work stage 6, workpieces (artificial organs or other organic or inorganic) materials) 7, pulse or RF power 14, DC power 15, target 16, change switch 17, gas supply 18 and bias power 19.

### EXAMPLE 8

Install workpieces (artificial organs) 7 on sample stage 6 in the vacuum chamber of the magnetron sputter-ion coating equipment. Use a piece of Ti—Ta (or Ti—Nb) alloy (or a piece of Ti embedded with Ta or Nb) as the sputter target 16. Pump out air from the vacuum chamber to pressure 10⁻⁴ Pa. Back fill the vacuum chamber of argon 0.01˜10 Pa. Heat workpieces 7. Open gas supply 18. Turn on switch 17 to the position of pulse potential 14 or direct potential 15 and apply a pulse or direct negative potential on the target 16. Argon plasma will be created and bombard on the target. Ti and Ta (or Ti and Nb) atoms will be sputtered out from the target and deposit on the workpieces 7 and form an alloy coating. During the coating process, the quality of the coating film can be improved by turning on the bias power 19 and applying a pulse or direct negative potential on the workpieces. This coating process can be performed using parameters listed in Table 10. The factors controlling the property of the film are: the atomic ratio of the Ta and Ti (or Ti and Nb) in the alloy target material or embedded target material 0.5:100˜10:100, direct potential −300˜−1,000 V, the width of the pulse sputter potential 1˜100 μs, frequency 5,000˜50,000 Hz and pulse sputter potential −300˜−1,000 V, power density 1˜15 W/cm², temperature of the workpieces is 100˜500° C., sputter processing time 0.01˜2 hour, sputter pressure 0.01˜10 Pa, the direct bias potential on the workpieces 0˜−3,000V, pulsed bias potential 0˜5,000V, frequency 1,000˜50,000 Hz, pulse width 1˜100 μs.

TABLE 10

| | Ta (Nb) | Pulse | Direct | Average | | | | | | Poltential | Frequency of | Pulse |
|---|---|---|---|---|---|---|---|---|---|---|---|---|
| | content in | potential | potential | power | Pulse | Pulse | Temper- | Treating | Argon | on sample | Pulse potential | width on |
| | the target | for sputter | for sputter | density | width | frequency | ature | time | pressure | stage | on workpiece | workpiece |
| Condition | (at. %) | (V) | (V) | (W/cm²) | (μs) | (Hz) | (° C.) | (hour) | (Pa) | (V) | (Hz) | (μs) |
| 1 | 1 | −400 | | 2 | 5 | 40,000 | 200 | 1.5 | 0.1 | −100 | 10,000 | 2 |
| 2 | 3 | −600 | | 3 | 10 | 20,000 | 300 | 0.8 | 0.2 | −300 | 20,000 | 10 |
| 3 | 8 | −1,000 | | 6 | 50 | 5,000 | 500 | 0.1 | 1 | −3,000 | 40,000 | 5 |
| 4 | 3 | | −500 | 12 | | | 250 | 0.6 | 0.2 | −500 | 8,000 | 20 |

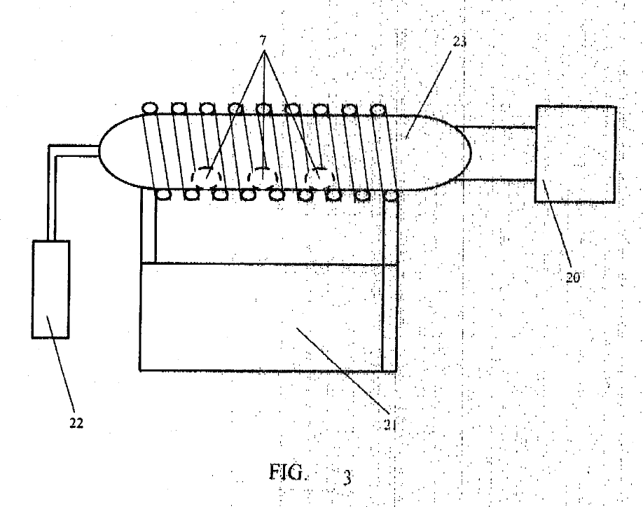

After coating, the workpieces can be oxidized by means of heat treatment or plasma treatment. The coating film will then change to be titanium oxide film containing tantalum (or niobium). FIG. 3 is the schematic drawing of the quartz vacuum furnace used for heat oxidization treatment or plasma oxidation in the present invention. The furnace consists of: workpieces 7, vacuum system 20, heating elements 21, gas filling system 22 and quartz tube 23. The working modes are:

A. Oxidization by Heating Treatment

Put the artificial 7 coating with Ti—Ta (or Ti—Nb) film in the quartz tube 23. Turn on the vacuum system 20 and vacuum the tube to 10⁻³ Pa. Turn on the heating elements 21 and heat the quartz tube to 400˜900° C. Open the gas filling system 22 and fill oxygen into the quartz tube to 0.1˜10 Pa. The film will be transform to titanium oxide containing tantalum (or niobium).

B. Oxidization by Plasma Treatment

Referring FIG. 1, install artificial organs 7 coated with Ti—Ta (or Ti—Nb) film on the sample stage 6 of PIII equipment. Vacuum the vacuum chamber to 10⁻⁴ Pa, then back fill the chamber with oxygen. Turn on power for RF discharge 9 (or MW discharge 10) to create oxygen plasma. The artificial organs are now immersed in the oxygen plasma environment. Heat artificial organs 7. Turn on the power of low pulse potential 12 and apply a pulse negative potential on the artificial organs 7. The coated metal film will transform to a titanium oxide film containing tantalum (or niobium). The process can be performed using parameters listed in Table 11. The factors controlling the properties of the film are: oxygen pressure 0.01˜10 Pa, the density of oxygen plasma 10⁸˜10¹² cm⁻³, temperature 100˜600° C., the amplitude of the negative pulse potential 0.2˜3 kV, repeat frequency 1,000˜20,000 Hz, pulse width 2˜200 μs, treating time 5˜120 min.

TABLE 11

| | Oxygen | Density of | Heating | Pulse | | Pulse | Pulse |
|---|---|---|---|---|---|---|---|
| | pressure | oxygen plasma | Temperature | potential | Treating time | frequency | width |
| Condition | (Pa) | (cm⁻³) | (° C.) | (kV) | (hour) | (Hz) | (μs) |
| 1 | 0.01 | 1 × 10⁸ | 200 | −0.2 | 2 | 5,000 | 100 |
| 2 | 0.2 | 3 × 10⁹ | 400 | −2 | 0.5 | 20,000 | 2 |
| 3 | 2 | 1 × 10¹¹ | 600 | −3 | 0.05 | 2,000 20 | |

### EXAMPLE 9

First, prepare a titanium nitride film containing tantalum (or niobium) and then transfer the nitride film by oxidization treatment.

Install artificial organs 7 on sample stage 6 in the vacuum chamber of the magnetron sputter-ion coating equipment. Use a piece of Ti—Ta (or Ti—Nb) alloy or a piece of Ti metal with Ta (or Nb) embedded inside, as the sputter target 16. Pump out air from the vacuum chamber to pressure 10⁻¹ Pa. Heat artificial organs 7. Open gas supply 18 and fill the vacuum chamber with some nitrogen and argon. Turn on switch 17 to the position of pulse potential 14 or direct potential 15 and apply a pulse or direct negative potential on target 16 to create argon and nitrogen plasmas. Under the effect of the negative potential, argon and nitrogen plasmas will bombard on the target and sputter out titanium and tantalum (or titanium and niobium) atoms. The titanium and tantalum (or titanium and niobium) atoms deposit on the artificial organs and form a titanium nitride film, containing tantalum (or niobium). During the coating process, the quality of the coating film can be improved by turning on bias power 19 and applying a pulse or direct negative potential on the sample stage 6. This process can be performed using parameters listed in Table 12 and Table 13. The factors controlling the property of the film are: the atomic ratio of the Ta and Ti (or Ti and Nb) in the target material 0.5:100˜10:100, the sputter potential −100˜−1,000 V, sputter current 0.05˜10 A, the temperature of the workpieces 100˜500° C. sputter time 0.1˜2 hour, sputter pressure 0.1˜2 Pa, the direct potential on the sample stage 0˜−1,000 V, pulse potential 0˜−5,000V, pulse width 1˜200 μs, frequency 5,000˜50,000 Hz. The artificial organs coated with titanium nitride film can be oxidized to produce a titanium oxide film containing tantalum (or niobium).

TABLE 12

| | Ta (Nb) | potential | Sputter | Tem- | | | Ni- | Potential |
|---|---|---|---|---|---|---|---|---|
| | content in | for | power | pera- | Sputter | Argon | trogen | on |
| Con- | the target | sputter | denaity | ture | time | Pressure | Pressure | sample |
| dition | (at. %) | (V) | (W/cm²) | ° C. | (hour) | (Pa) | (Pa) | stage (V) |
| 1 | 0.5 | −300 | 3 | 200 | 1 | 0.8 | 0.8 | −200 |
| 2 | 3 | −600 | 4 | 300 | 0.8 | 0.5 | 0.4 | −300 |
| 3 | 10 | −1,000 | 8 | 500 | 0.2 | 0.3 | 0.2 | −600 |

TABLE 13

| | Ta (Nb) | Pulse | Sputte | | | | | Ni- | Pulse | Pluse | Frequency of | |
|---|---|---|---|---|---|---|---|---|---|---|---|---|
| | content | Potential | power | Pulse | Pulse | Sputter | Argon | trogen | potential | width on | Pluse potential | |
| | in target | for | density | frequency | width | time | pressure | pressure | on sample | workpieces | on workpieces | temperature |
| Condition | (at. %) | sputter (V) | (W/cm²) | (Hz) | (μs) | (hour) | (Pa) | (Pa) | stage (V) | (μs) | (Hz) | (° C.) |
| 1 | 1.5 | −400 | 3 | 10,000 | 20 | 1 | 0.8 | 0.8 | −1,000 | 5 | 5,000 | 350 |
| 2 | 3 | −600 | 5 | 40,000 | 2 | 0.8 | 0.5 | 0.4 | −4,000 | 2 | 8,000 | 300 |

### EXAMPLE 10

Production of Titanium Oxide Film Containing Tantalum or Niobium Using Pulse Sputter Technique

Use a piece of Ti—Ta (or Ti—Nb) alloy, or a piece of Ti metal with Ta (or Nb) embedded inside, as sputter target 16 on the magnetron sputter-ion coating equipment. Install artificial organs 7 on sample stage 6 in the vacuum chamber of the magnetron sputter-ion coating equipment. Pump out air from the vacuum chamber to pressure 1×10⁻⁴ Pa. Heat artificial organs 7. Open gas supply 18 and fill the vacuum chamber with argon and oxygen to both 0.01˜10 Pa. Turn on switch 17 to the position of pulse negative potential 14 and apply a pulse potential on the target 16 to create argon and oxygen plasmas. Under the effect of the negative potential, argon and oxygen plasmas will bombard on the target and sputter out titanium and tantalum (or titanium and niobium) atoms. The titanium and tantalum (or titanium and niobium) atoms deposit on the artificial organs and combine oxygen atoms to form a titanium oxide film, containing tantalum (or niobium). During the process, the quality of the film can be improved by turning on bias power 19 and applying a pulse negative potential on the workpieces. This process can be performed using parameters listed in Table 14. The factors controlling the property of the film are: the atomic ratio of the Ta and Ti (or Ti and Nb) in the target material 0.5:100˜10:100, the sputter potential on the target −300˜−1,000 V, frequency 10,000˜50,000 Hz, pulse width 1˜60 μs, sputter power density 1˜15 W/cm², the temperature of the workpiece 20˜500° C., sputter time 0.1˜2 hour, argon pressure 0.01˜2 Pa, oxygen pressure 0.01˜2 Pa, the pulse potential on the sample stage 0˜−5,000 V, pulse width 1˜100 μs, frequency 5,000˜50,000 Hz, the rotation speed of the sample stage 1˜100 turn/min.

TABLE 14

| | Ta (Nb) | Pulse | Sputte | | | | | Pulse | Frequency of | Pluse |
|---|---|---|---|---|---|---|---|---|---|---|
| | content in | Potential | power | Heating | Sputter | Argon | Oxygen | potential | Pluse potential | width on |
| | the target | for | density | temperature | time | pressure | pressure | on sample | on sample | sample |
| Condition | (at. %) | sputter (V) | (W/cm²) | (° C.) | (hour) | (Pa) | (Pa) | stage (V) | stage (Hz) | satge (μs) |
| 1 | 0.5 | −300 | 2 | 100 | 1.5 | 1 | 0.5 | −3,000 | 50,000 | 2 |
| 2 | 3 | −600 | 5 | 300 | 0.8 | 1.5 | 1 | −1,000 | 5,000 | 100 |
| 3 | 10 | −1,000 | 8 | 500 | 0.2 | 2 | 2 | −300 | 100 | 500 |

### EXAMPLE 11

Production of Titanium Oxide Film Containing Tantalum or Niobium Using RF Discharge Sputter Technique and Ta₂O₅—TiO₂ (or Nb₂O₅—TiO₂) Ceramic Target Material

Use a piece of Ta₂O₅—TiO₂ (or Nb₂O₅—TiO₂) ceramic target material as sputter target 16 on the magnetron sputter-ion coating equipment. Install artificial organs 7 on sample 6 in the vacuum chamber of the magnetron sputter-ion coating equipment. Pump out air from the vacuum chamber to pressure 1×10⁻⁴ Pa. Heat artificial organs 7. Open gas supply 18 and fill the vacuum chamber with argon of 0.01˜10 Pa. Turn on switch 17 to the position of RF discharge 14 and apply a RF discharge potential on target 16 to create argon plasma. Argon plasma will bombard on the target and sputter out titanium, tantalum and oxygen (or titanium, niobium and oxygen) atoms. The titanium, tantalum and oxygen (or titanium, niobium and oxygen) atoms deposit on the artificial organs 7 and form a titanium oxide film containing tantalum (or niobium). During the process, the quality of the film can be improved by turning on bias power 19 and applying a negative potential on the sample stage 6. The process of producing a titanium oxide film containing tantalum (or niobium) using RF discharge sputter technique can be performed using parameters listed in Table 15. The factors controlling the property of the film are: The discharge power 1˜10 W/cm², argon pressure 0.01˜10 Pa, the temperature of the workpieces 100˜600° C., sputter time 0.1˜3 hour.

TABLE 15

| | Ta₂O₅ (Nb₂O₅) | Heating | RF | Argon | Sputter | Potential on |
|---|---|---|---|---|---|---|
| | contentin the | temperature | Discharge | prossure | time | workpiece |
| Condition | target (%) | (° C.) | power (W) | (Pa) | (hour) | (V) |
| 1 | 0.3 | 200 | 200 | 5 | 2 | 0 |
| 2 | 1.5 | 400 | 800 | 0.5 | 1 | −300 |
| 3 | 5 | 600 | 2,500 | 0.05 | 0.5 | −500 |

FIG. 4 shows adhesion of platelets on a film produced by the resent invention (a) and on TLIC (b) after in vitro test. The number of platelets (the white particles) in (a) is obviously less than that in (b), indicating that the blood compatibility of the material produced by the present invention is obviously superior to that of LTIC.

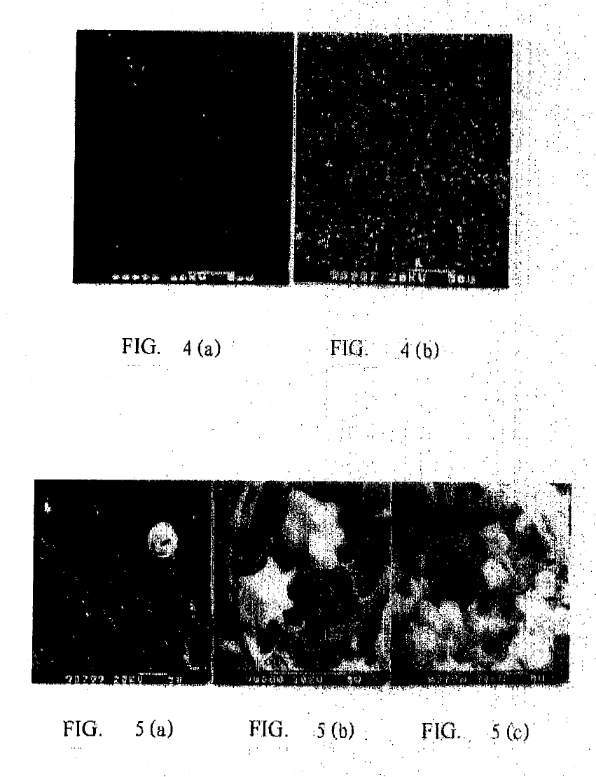

FIG. 5 (a) shows in vivo test result of blood cell adhesion on a film produced by the present invention; (b) and (c) show in vivo test result of blood cell adhesion on the surface of LTIC. The in vivo test is to implant a piece of Ti metal and a piece of LTIC in the right atrium of a test animal (a dog) for two weeks during which the test animal lived in the normal life and no anti-thromboembolic drugs are used. The Ti metal is coated with a TiO₂ film prepared by the present invention. After two weeks time, the test pieces are taken out from the test animal by surgery operation under anesthetic condition. The micrographs are taken using scanning electron microscopy at the Ti metal and LTIC been critical dried. On the micrographs it can be seen that there are only few erythrocytes in the original normal shape, and no thrombus on the surface modified Ti metal (FIG. 5 (a)), but on LTIC the erythrocytes have been deformed and destroyed seriously (FIG. 5 (b)), and thrombus have formed (FIG. 5 (c)).

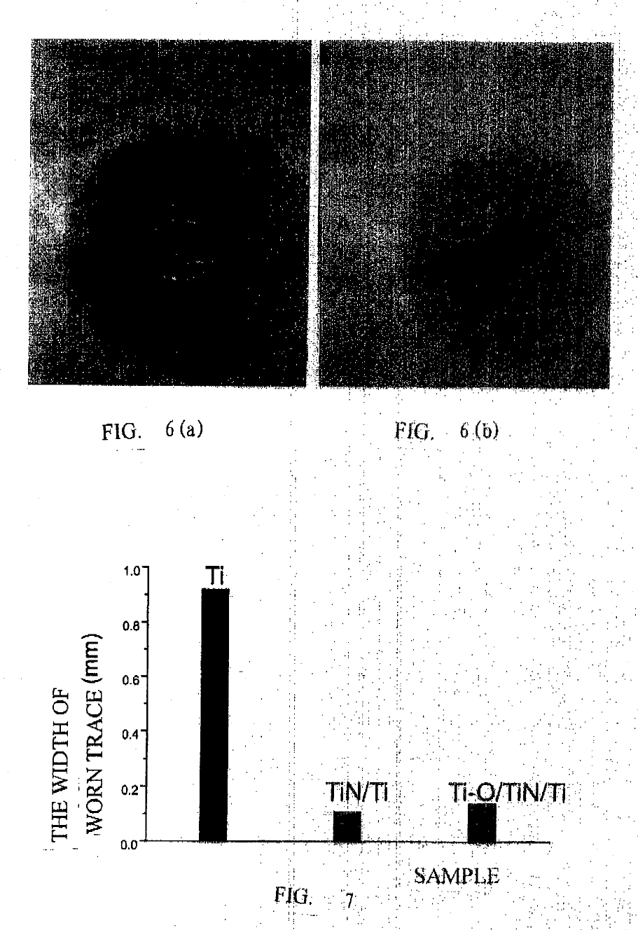

FIG. 6 is the comparison of in vivo test results of thrombus formation on commercial artificial heart valve cages with and without modification by the present invention. The in vivo test is done by implant commercial artificial heart valve cages, one with a surface film prepared by the present invention and the other without, into the test animal (a dog) body. The artificial heart valve cages were hanged in right atrium of the dog's heart. During the test, the test animal lived in the normal life and no anti-thromboembolic drugs are used. After three months time, the heart valve cages are taken out from the test animal by surgery operation under anesthetic condition. FIG. 6 (a) shows the formation of thrombus on the artificial heart valve cage with a film surface prepared by the present invention. FIG. 6 (b) shows the formation of thrombus on the artificial heart valve cage which has no been treated. It can be seen from the micrographs that only very few thrombus formed on the modified hear valve cage surface, while on the heart valve cage without treatment, thrombus has formed all over the cage surface.

FIG. 7 is comparison in wear resistances of titanium materials for artificial heat valves with and without surface modification using the present invention. It can be seen that the titanium material with modified surface using the present invention is much superior to the material without modification in wear resistance.

As said above, surface films prepared by the present invention are obviously superior to the materials of prior art in both blood compatibility and wear resistance, which consists of basis of claims of the present invention. Although certain presently preferred embodiments of the present invention have been described specifically in the examples, the present invention should not be considered to be only as described herein. It will be apparent to those skilled in the art to which the invention pertains that variations and modifications of the various embodiments shown and described herein may be made without departing from the spirit and scope of the invention.

1. An omni-directional surface modification method to produce a TiO₂₋ₓ films on inorganic and organic materials by means of plasma immersion ion implantation (PIII) process. The method includes the following procedures:
(a) Introduce oxygen into the vacuum chamber, and oxygen being in gaseous or plasma state in the vacuum chamber;
(b) By means of the metal arc plasma source to create titanium plasma and introduce the titanium plasma into the vacuum chamber, and titanium atoms will deposit on the surfaces of the materials mentioned in this term;
(c) Apply a negative pulse potential on the workpieces. The pulse potential has frequency of 500˜50,000 Hz, pulse potential amplitude 0.1˜10 kV and pulse width 1˜200 μs.

2. The said oxygen pressure as recited in claim 1 is about 10⁻³˜1 Pa. If oxygen exists in plasma state in the vacuum chamber, its density is 10⁸˜10¹²/cm³.

3. The said density of titanium metal plasma as recited in claim 2 is about 10⁸˜10¹²/cm³, the titanium deposition rate about 0.1˜1 nm/s.

4. The said TiO₂₋ₓ film as recited in claim 1 has a thickness of 0.05˜5 μm.

5. The said TiO₂₋ₓ film as recited in claim 1 has a rutile crystal structure, chemical composition TiO₂₋ₓ (where x=0˜0.35).

6. The surface that is synthesized with the said method as recited in claim 1 is of artificial organs.

7. The surface that is synthesized by the said method as recited in claim 1 is of devices for implanting into human bodies and contacting blood.

8. An omni-directional surface modification method to produce TiO₂₋ₓ/Ti—N—O/TiN gradient films on surfaces of inorganic and organic materials by means of plasma immersion ion implantation (PIII) process. The method includes following procedures:
(a) Introduce nitrogen into the vacuum chamber, and nitrogen being in gaseous or plasma state in the vacuum chamber;
(b) By means of the metal arc plasma source to create titanium plasma and introduce the titanium plasma into the vacuum chamber, and titanium and nitrogen atoms will bombard and deposit on the surfaces of the materials mentioned in this term to form a TiN substrate;
(c) Decrease the nitrogen pressure at a rate of 10⁻³˜10⁻² Pa/min, and increase oxygen pressure at a rate of 10⁻³˜10⁻² Pa/min to produce a transition layer with gradual decreasing nitrogen concentration and increasing oxygen content;
(d) Maintain only oxygen (oxygen is O₂ or oxygen plasma) and titanium plasma in the vacuum chamber to produce a TiO₂₋ₓ (x is 0˜0.35) layer on top of the surface.

9. The said nitrogen pressure as recited in claim 8 is about 10⁻³˜1 Pa. If nitrogen is in plasma state in the vacuum chamber, its density is 10⁸˜10¹²/cm³.

10. The said oxygen pressure as recited in claim 8 is about 10⁻³˜1 Pa. If oxygen exists in plasma in the vacuum chamber, its density is 10⁸˜10¹²/cm³.

11. The said density of titanium metal plasma as recited in claim 8 is about 10⁸˜10¹²/cm³.

12. The surface that is synthesized by the said method as recited in claim 8 is of artificial organs.

13. The surface that is synthesized with the said method as recited in claim 8 is of devices for implanting into human bodies and contacting blood.

14. The process as recited in claim 1 also includes the following procedures:
(e) Introduce hydrogen into the vacuum chamber. Hydrogen exists in the vacuum chamber as hydrogen plasma;
(f) Apply a DC potential of −0.2˜5 kV, discharge current 0.1˜5A, or pulse potential of frequency of 5,000˜50,000 Hz, pulse width 1˜200 μs, average current of 0.1˜5A on the said workpiece;
(g) Treat the workpiece at temperature of 100˜600° C. for 0.1˜2 h to form hydrogen-doped titanium oxide films;
(h) Anneal the workpieces at temperature of 200˜400° C. for 0.1˜1 h in 10⁻⁴˜10⁻¹ Pa vacuum.

15. The hydrogen content in the surface film that is synthesized with the said method as recited in claim 14 is from 10 at. %˜35 at. %, with the best value being 20 at. %.

16. The hydrogen pressure of the said process as recited in claim 14 step (e) is about 10⁻³˜10 Pa, and the hydrogen plasma density is about 10⁸˜10¹² cm⁻³.

17. The surface that is modified with the said process as recited in claim 14 is of artificial organs.

18. The surface that is modified with the said process as recited in claim 14 is of devices for implanting into human bodies and contacting blood.

19. The process as recited in claim 8 also includes the following procedures:
(e) Introduce hydrogen into a vacuum chamber. Hydrogen exists in the vacuum chamber as hydrogen plasma;
(f) Apply pulse potential with the frequency 50˜20,000 Hz, amplitude 1˜100 kV, and pulse width 1˜200 μs on the said workpieces to form hydrogen-doped titanium oxide films;
(g) Anneal the workpieces at temperature of 200˜400° C. for 0.1˜2 h at 10⁻⁴˜10⁻¹ Pa vacuum.

20. In the process as recited in claim 19, the hydrogen content in the surface film is from 10 at. %˜35 at. %, with the best value being 20 at. %.

21. In the process as recited in claim 19, procedure (e), hydrogen pressure is about 10⁻³˜10 Pa, hydrogen plasma density is about 10⁸˜10¹² cm⁻³, and the implantation dosage of hydrogen is 10¹⁵˜1.2×10¹⁸ atom/cm³.

22. The surface that is modified with the said process as recited in claim 19 is of artificial organs.

23. The surface modified with the said process as recited in claim 19 is of devices for implanting into human bodies and contacting blood.

24. The process as recited in claim 19 also includes the following procedures:
(e) Introduce hydrogen into the vacuum chamber. Hydrogen exists in the vacuum chamber as hydrogen plasma;
(e) Apply a pulse potential of the frequency of 50 to 20,000 Hz on the said workpieces;
(g) Implant hydrogen ions into the workpieces for about 0.1˜2 h;
(h) Adjust the potential to a lower level to implant hydrogen ions for about 0.1˜2 hours;
(i) Adjust the pulse potential to an even lower level to implant hydrogen ions for about 0.1˜2 hours;
(j) Adjust the pulse voltage again to an even lower value to implant hydrogen ions for about 0.1˜2 hours;
(k) Repeat steps (g) to (j) about 2˜10 times, with the best being 3˜4 times, to form hydrogen-doped titanium oxide films with a homogeneously distribution of hydrogen atoms in the depth direction. The hydrogen content in the surface film is about 10%-35%, with the best being 20%;
(l) Anneal the workpieces at temperature about 100˜400° C. for 0.1 to 2 h at 10⁻⁴ to 10⁻¹ Pa vacuum.

25. In the said process as recited in claim 24, hydrogen pressure in procedure (e) is about 10⁻²˜10 Pa, hydrogen plasma density is about 10⁸˜10¹² cm⁻³, and implantation dosage of hydrogen is 10¹⁶˜10¹⁸ atom/cm³.

26. In the said process as recited in claim 24, the pulse potential in procedure (f) is about −60˜100 kV.

27. In the said process as recited in claim 24, the pulse potential in procedure (h) is about −30˜−60 kV.

28. In the said process as recited in claim 24, the pulse potential in procedure (i) is about −10˜−30 kV.

29. In the said process as recited in claim 24, the pulse potential in procedure (j) is about −0.1˜−10 kV, with the best value being −7 kV.

30. The surface that is obtained with the said process as recited in claim 24 is of artificial organs.

31. The surface that is modified with the said process as recited in claim 24 is of devices for implanting into human bodies and contacting blood.

32. As recited in claim 1, the said process also includes the following procedures:
(d) Implant tantalum or niobium ions into TiO₂₋ₓ film using metal cathode plasma source;
(e) Apply a pulse high potential of −1˜−100 kV, frequency of 100˜20,000 Hz, pulse width 1 to 200 μs to the workpieces;
(f) Anneal the workpieces at temperature of 100˜800° C. in 10⁻⁴ to 10⁻¹ Pa vacuum for 0.1˜2 h.

33. In process (d) of the said process of claim 32, the tantalum or niobium plasma density is 10⁸˜10¹² cm⁻³, the implantation dosage of tantalum or niobium is about 10¹⁵˜5×10¹⁷ atom/cm³.

34. In procedure (d) of the said process of claim 32, the ratio of tantalum plasma to titanium plasma, or of niobium plasma to titanium plasma, is 0.5:100˜10:100. The atomic ratio of Ta or Nb to Ti in the surface film is about 0.5:100˜10:100.

35. The surfaces that are obtained with the said process 32 are of artificial organs.

36. The surfaces that are obtained with the said process 32 are of devices for implanting into human bodies and contacting blood.

37. An omni-directional surface modification method to produce TiO₂₋ₓ films containing Ta or Nb on inorganic and organic materials by means of plasma immersion ion implantation (PIII) process. The method includes the following procedures:
(a) Introduce oxygen into a vacuum chamber, and oxygen exists as gas or plasma in the vacuum chamber;
(b) Use a Ti—Ta or Ti—Nb alloy as the cathode material. Introduce titanium and tantalum or titanium and niobium plasmas into the vacuum chamber and deposit titanium oxide films, containing Ta or Nb, on the said workpieces;
(c) Apply a negative pulse potential of frequency of 100˜20,000 Hz, magnitude of 0.01˜20 kV, pulse width 1˜200 μs, on the workpieces.

38. In the said process as recited in claim 37, oxygen pressure is about 10⁻³˜1 Pa, oxygen exist as oxygen plasma, and the density of oxygen plasma is 10⁸˜10¹² cm⁻³.

39. In the said process as recited in claim 37, the atomic ratio of Ta:Ti or Nb:Ti in the cathode material is about 0.5:100˜10:100, the plasma densities of Ti—Ta or Ti—Nb are about 10⁸˜10¹² cm⁻³, and the deposition rate of Ti—Ta—O or Ti—Nb—O film is about 0.1˜1 nm/s.

40. In the said process as recited in claim 37, the thickness of Ti—Ta—O or Ti—Nb—O film is about 0.05˜5 μm.

41. Obtained with the said process as recited in claim 37, the structure of Ti—Ta—O or Ti—Nb—O film has a rutile crystal structure, and the atom ratio of Nb:Ti or Ta:Ti is about 0.5:100˜10:100.

42. The surface that is treated with the said process as recited in claim 37 is of an artificial organ.

43. The surface that is treated with the said process as recited in claim 37 is of a device that is for implanting into human bodies and contacting blood.

44. An omni-directional surface modification method to produce titanium oxide film containing Ta or Nb on inorganic and organic materials by means of sputtering. The method includes the following procedures:
(a) Use Ti—Nb or Ti—Ta alloy target or embedded target. Deposit Ti—Nb or Ti—Ta allow films by sputtering on the said workpieces surface;
(b) Oxidize the alloy films to form Ta— or Nb-doped titanium oxide films, with the content ratio of Ta:Ti or Nb:Ti being about 0.5:100˜10:100.

45. In procedure (a) of the said process as recited in claim 44, a pulse potential of 0˜−5,000 V, pulse width 1˜100 μs and frequency 5,000˜50,000 Hz is applied on the workpieces. Use argon plasma, a pulse sputtering potential of −300˜−1,000 V and pulse width 1˜100 μs, sputtering power density 1˜15 W/cm² to sputter atoms from the target. The temperature of the workpieces is about 100˜500° C., argon sputter pressure of is 0.05˜2 Pa, and sputtering time is 0.1˜2 h.

46. In procedure (b) of the said process as recited in claim 44, the oxygen pressure is about 0.1˜10 Pa, temperature is 400˜900° C., and oxidation time is 10 min˜2 h.

47. In procedure (b) of the said process as recited in claim 44, the pulse potential applied on the workpieces has a frequency 1,000˜20,000 Hz and potential value −0.2˜−3 kV.

48. In the said process as recited in claim 44, the atomic ratio of Ta:Ti or Nb:Ti in the Ti—Ta or Ti—Nb alloy target material is about 0.5:100˜10:100.

49. An omni-directional surface modification method to produce titanium oxide films containing Ta or Nb on inorganic and organic materials by means of sputtering and oxidation. The method includes the following procedures:
(a) Using Ti—Nb or Ti—Ta alloy or embedded target. Deposit titanium nitride films containing Ta or Nb, on the workpiece surface;
(b) Oxidize the nitride films to form titanium oxide films containing tantalum or niobium, with the atomic ratio of Ta:Ti or Nb:Ti being about 0.5:100˜10:100.

50. In procedure (a) of the said process as recited in claim 49, a pulse potential of 0˜−5,000 V, pulse width 1˜200 μs and frequency 5,000˜50,000 Hz is applied on the workpiece. Use argon plasma, a pulse sputtering potential of −300˜−1,000 V and pulse width 1˜100 μs, sputtering power density 1˜15 W/cm² to sputter atoms from the target. The temperature of the workpieces is about 20˜500° C., sputter pressure of is 0.05˜2 Pa, and sputtering time is 0.1˜2 h.

51. In procedure (a) of the said process as recited in claim 49, the atomic ratio of Ta:Ti or Nb:Ti in the Ti—Ta or Ti—Nb alloy target is about 0.5:100˜10:100.

52. The surface that is treated with the said process as recited in claim 49 is of artificial organs.

53. The surface that is modified with the said process as recited in claim 49 is of devices for implanting into human bodies and contacting blood.

54. An omni-directional surface modification method to produce titanium oxide films containing Ta or Nb on inorganic and organic materials by means of sputtering. The method includes the following procedures:
(a) Use a Ti—Nb or Ti—Ta alloy or embedded target. Deposit titanium oxide films containing Ta or Nb;
(b) Apply a pulse potential of 0˜−5,000 V, pulse width of 1˜100 μs and frequency of 5,000˜50,000 Hz on the workpieces. Use argon and oxygen, at a pulse potential of −300˜−1,000 V, pulse width of 1˜60 μs, frequency 10000˜50000 Hz, power density 1˜15 w/cm², sputtering pressure 0.01˜10 Pa, temperature about 20˜500° C., to sputter and deposit for 0.1˜2 hour. The atomic ratio of Ta:Ti or Nb:Ti in the target alloy is about 0.5:100˜10:100.

55. The surface that is treated with the said process as recited in claim 54 is of artificial organs.

56. The surface that is treated with the said process as recited in claim 54 is of devices for implanting into human bodies and contacting blood.

57. An omni-directional surface modification method to produce TiO₂/Ti—O—N/TiN gradient films containing Ta or Nb on inorganic and organic materials by means of sputtering. The method includes the following procedures:
(a) Use a Ti—Nb or Ti—Ta alloy target or embedded target. Deposit titanium nitride films containing Ta or Nb by sputter method;
(b) Synthesize Ti—N—O gradient transition films containing Ta or Nb;
(c) Synthesize titanium oxide films containing Ta or Nb.

58. In procedure (a) of the said process of claim 57, a pulse potential of 0˜−5,000V, pulse width of 1˜100 μs and frequency of 1,000˜20,000 Hz, is applied on the workpieces. Use argon and oxygen, at a pulse potential of −300˜−1,000 V, pulse width of 1˜100 μs, average sputter power density 1˜15 w/cm², sputtering pressure 0.01˜10 Pa, temperature about 20˜500° C., to sputter and deposit for 0.1˜2 hour.

59. In procedure (b) of claim 57 of the said process, nitrogen pressure decreases at a rate of about 0.001˜0.01 Pa/min., oxygen pressure increases at a rate of about 0.001˜0.01 Pa/min., the pressure of argon is about 0.01˜10 Pa, the other parameters are the same as in procedure (a).

60. In procedure (c) of claim 57 of the said process, the gas in the vacuum chamber are argon and oxygen, the parameters are the same as in procedure (a).

61. In claim 57 of the same process, the atomic ratio of Ta or Nb and Ti in the alloy target is about 0.5:100˜10:100.

62. The surface that is synthesized with the said method as recited in claim 57 is of artificial organs.

63. The surface that is synthesized by the said method as recited in claim 57 is of devices for implanting into human bodies and contacting blood.

64. An omni-directional surface modification method to produce TiO₂/Ti—O—N/TiN gradient films on inorganic and organic materials by means of sputtering, using ceramic target material. The method is: Use a Ta₂O₅—TiO₂ or Nb₂O₅—TiO₂ ceramic as the target material, RF discharge sputter power density 1˜10 w/cm², argon pressure 10²˜10 Pa in the vacuum chamber, temperature about 20˜600° C., potential 0˜−1,000V, to sputter and deposit for 0.1˜3 hour. In the target material the molecular ratio of content of Ta₂O₅:TiO₂ or Nb₂O₅:TiO₂ is 0.05:100˜5:100.

65. The surface that is synthesized with the said method as recited in claim 64 is of artificial organs.

66. The surface that is synthesized by the said method as recited in claim 64 is of devices for implanting into human bodies and contacting blood.
